(19) United States
(12) Patent Application Publication
(10) Pub. No.: US 2022/0241340 A1
(43) Pub. Date: Aug. 4, 2022

(54) COMPOSITION OF PHARMACEUTICAL CARRIER SOLUTION FOR MESENCHYMAL STEM CELLS AND USE OF THE SAME

(71) Applicant: BAYLX, INC., Irvine, CA (US)

(72) Inventors: Wenbin LIAO, Irvine, CA (US); Yongjun LIU, Irvine, CA (US); Linan LIU, Irvine, CA (US)

(21) Appl. No.: 17/629,686

(22) PCT Filed: Aug. 6, 2020
(86) PCT No.: PCT/US2020/045262

§ 371 Date: Jan. 24, 2022

Related U.S. Application Data
(60) Provisional application No. 62/884,824, filed on Aug. 9, 2019.

(51) Int. Cl. A61K 35/28; A61P 37/00; A61K 47/42

(52) U.S. Cl. CPC A61K 35/28; A61K 47/42; A61P 37/00

(57) ABSTRACT

The invention provides compositions of a carrier solution to resuspend mesenchymal stem cells (MSC), methods to formulate an MSC-containing pharmaceutical composition for the treatment of medical diseases. Practicing these methods and composition will greatly preserve the cell viability, identity and biological function, and substantially slow down the cell death and function loss during preparation, storage or shipping, before the administration to a recipient.

The present invention claims priority to U.S. Provisional Application No. 62/884,824, filed on Aug. 9, 2019, which is incorporated by reference for all purposes as if fully set forth herein.

### TECHNICAL FIELD

The present invention relates to compositions of a carrier solution for mesenchymal stem cells (MSC), the method to formulate MSC product with the carrier solution and the use of the same.

### BACKGROUND

MSC have great potential for the treatment of a series of medical disorders. In many circumstances, fresh MSC products containing living cells are manufactured and temporarily stored before they are administrated systemically or in a local site to achieve a clinical efficacy. Therefore, it is crucial to maintain the cell viability, identity, biological functions, specific cell status (e.g., Single cell suspension) during the storage or shipping before administration to a recipient. For this purpose, the MSC are usually required to be formulated in a liquid solution with or without supplementing with other excipients to preserve cell viability and functions for an optimal clinical efficacy in patients. Pharmaceutically acceptable carrier solutions for medical products have been described previously, for example, Remington's Pharmaceutical Science (18th Ed., Gennaro, Mack Publishing Co., Easton, Pa., 1990) and the Handbook of Pharmaceutical Excipients (4th Ed., Rowe et al. Pharmaceutical Press, Washington, D.C.) However, MSC-based cellular products are different from the traditional chemicals or molecules based medicines and have unique requirements for carrier solutions as mentioned above, thus an optimal composition of the carrier solutions specifically for MSC is yet to be developed.

### SUMMARY

This invention relates compositions of carrier solution to resuspend umbilical cord or other tissue derived mesenchymal stem cells, and a method to formulate MSC-containing pharmaceutical product with the carrier solution. More particularly, the composition of the carrier solution including three components: a balanced salt solution, a low molecular weight heparin and a human serum albumin. The method to formulate MSC-containing pharmaceutical product includes re-suspension of MSC in the said carrier solution to achieve a single cell suspension at a desired cell concentration between 1×10⁵ to 1×10⁷ cells per milliliter. Such MSC-containing pharmaceutical product can greatly preserve the cell viability, identity and biological function, and substantially slow down the cell death and function loss during storage or shipping before the administration to a recipient. Use of the composition and methods can achieve optimal therapeutic effects in certain medical conditions.

Mesenchymal stem cells (MSC) are adult stem cells originated from mesoderm (Crisan et al. 2008). Recent evidence have demonstrated that MSC are promising pharmaceutical candidate for the treatment of many medical diseases (Wang et al. 2016; Wang et al. 2013). In many circumstances, MSC are cryopreserved in DMSO containing liquid and directly administered into a recipient with or without washing and re-suspension. Most recently, it has been reported that fresh living cells product where cells are directly harvested from cell culture without cryopreservation before final formulation may be more therapeutically effective with less immunogenic reactions in vivo, as compared to product of cryopreserved cells (Chinnadurai et al. 2016; Moll et al. 2014; Francois et al. 2012). To achieve optimal pharmaceutical effects with cell product, it is reasonable to administer product of fresh cells due to the advantages of higher viability of cells, maintained cell identity and surface marker expression and lower complement activation for fresh cells versus cryopreserved cells.

A carrier solution is required to resuspend MSC for pharmaceutical use via systemic and/or local administration, and more importantly to preserve the viability and biological functions of the living MSC before administration. After formulation, there are several tests need to be done immediately (quality control) to release a fresh MSC product. Also the released product usually needs to be shipped from manufacturing facility to clinical sites for administration. Thus it is important to find out an optimal composition of the carrier solution to maintain the quality of MSC during the storage.

In several embodiments, there are provided compositions of the carrier solution comprising a type of balanced salt solution supplemented with other excipients that are important to maintain the viability and functions of living MSC. Also provided are methods of preparing a MSC product by resuspending the MSC in the carrier solution and filling the resuspended MSC-carrier solution mixture into a sterile infusion bag, and use of the MSC product as therapies for, including but not limited to autoimmune diseases, neural diseases, liver diseases or cancer.

In several embodiments, fresh living MSC resuspended in the provided compositions of the carrier solution comprising a type of balanced salt solution supplemented with other excipients have higher viability and more robust biological functions after storage in a specific conditions when compared to a control (e.g., Balanced salt solution without supplements).

In several embodiments, the composition of a carrier solution comprising at least three components: a type of balanced salt solution, a low molecular weight heparin and human serum albumin.

In several embodiments, the balanced salt solution is, but not limited to, lactated Ringer's solution, multiple electrolyte injection such as Plasma-Lyte A or 0.9% saline (sodium chloride).

In several embodiments, the low molecular weight heparin can be low molecular weight heparin sodium or low molecular weight heparin calcium, and the final concentration of heparin is between 5 IU/mL to 100 IU/mL.

In several embodiments, the final concentration of human serum albumin is between 0.5% to 5% (V/V).

In several embodiments, there are provided methods of preparing a finished MSC pharmaceutical product by resuspending the MSC in the carrier solution with said compositions and filling the resuspended MSC-carrier solution mixture into a sterile infusion bag.

The MSC tissue sources comprise preferably human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

Other examples of the MSC tissue sources include, but are not limited to, bone marrow, adipose tissues, blood, amniotic fluid, dental pulp and placenta.

In several embodiments, the concentration of MSC in the finished MSC pharmaceutical products is between 1×10⁵ to 1×10⁷ cells per milliliter.

In several embodiments, the finished MSC pharmaceutical products are stored at 2-8° C. and may be provided as therapies to a patient suffering from a medical disease.

In several embodiments, the finished MSC pharmaceutical products may be used to treat autoimmune diseases or liver diseases, or cancer.

In several embodiments, the finished MSC pharmaceutical products may treat various autoimmune diseases. These diseases may comprise Crohns' diseases, ulcerative colitis, multiple sclerosis, rheumatoid arthritis, systemic lupus erythematosus, autoimmune pancreatitis, Type 1 diabetes, systemic sclerosis.

In several embodiments, the finished MSC pharmaceutical products may treat various liver diseases. These liver diseases may comprise liver failure, cirrhosis, hepatic steatosis, hepatitis.

In several embodiments, the finished MSC pharmaceutical products may treat various cancers. These cancers may comprise lung cancer, breast cancer, liver cancer, gastrointestinal cancer, brain cancer, hematologic malignancy.

### BRIEF DESCRIPTION OF THE DRAWINGS

FIGS. 1A-1B show the cell viability (%) of mesenchymal stem cells (MSC) formulated in carrier solutions containing different concentrations (0%, 1%, and 2%) of human serum albumin (HSA) at 24 hours, 48 hours and 72 hours after formulation. Provided are (A) the table and (B) the graph indicating the viability of MSC was significant higher in the presence of HSA than that without HSA in the carrier solution. Mean±SD. HSA 0% vs. 1%: ^{####}p<0.0001. HSA 0% vs. 2%: ****p<0.0001. HSA 1% vs. 2%: ^{$$}p<0.01.

FIGS. 2A-2B show the MSC population (%) per flow cytometric event after formulated in different carrier solutions for 24 hours. Provided are the (A) table and (B) the graph indicating that the MSC population per event was significantly higher in lactated Ringer's solution with heparin sodium (*p<0.05) or Plasma-Lyte A with heparin calcium (****p<0.0001) compared to that in Plasma-Lyte A with heparin sodium. The MSC population (%) tended to increase as the calcium concentration went up. No significant difference was observed between cell suspension in lactated Ringer's solution with heparin sodium and Plasma-Lyte A with heparin calcium. *p<0.05, and ****p<0.0001. n.s., not significant. All carrier solutions contain 2% HSA.

FIG. 3 shows the MSC population (%) per flow cytometric event after formulated in Plasma-Lyte A with different concentrations of heparin calcium for 24 hours. Provided are representative flow cytometric plots (FSC-A vs. SSC-A) indicating that the MSC population (%) tended to grow as the level of heparin sodium increased in the carrier solution. All carrier solutions contain 2% HSA.

FIG. 4 shows the MSC population (%) per flow cytometric event after formulated in Plasma-Lyte A with different concentrations of heparin sodium for 24 hours. Provided are representative flow cytometric plots (FSC-A vs. SSC-A) indicating that the MSC population (%) remained at same level with different concentrations of heparin sodium in the carrier solution. All carrier solutions contain 2% HSA.

FIG. 5 shows the MSC population (%) per flow cytometric event after formulated in lactated Ringer's solution with different concentrations of heparin sodium for 24 hours. Provided are representative flow cytometric plots (FSC-A vs. SSC-A) indicating that the MSC population (%) remained at same level with different concentrations of heparin sodium in the carrier solution. All carrier solutions contain 2% HSA.

FIGS. 6A-6B show no significant difference on single cell population (%) and cell viability (%) was observed between cell suspensions in lactated Ringer's solution with heparin sodium and Plasma-Lyte A with heparin calcium. Provided are the (A) graph of single cell population (%) and (B) the graph of cell viability (%). All carrier solutions contain 2% HSA.

### DETAILED DESCRIPTION

This invention relates compositions of carrier solution to resuspend umbilical cord tissue mesenchymal stem cells, and methods to formulate MSC-containing pharmaceutical product with the carrier solution. More particularly, the composition of the carrier solution including three components: a balanced salt solution, a low molecular weight heparin and a human serum albumin. The method to formulate MSC-containing pharmaceutical product includes re-suspension of MSC in the said carrier solution to achieve a single cell suspension at a desired cell concentration between 1×10⁵ to 1×10⁷ cells per milliliter. Such MSC-containing pharmaceutical product can greatly preserve the cell viability, identity and biological function, and substantially slow down the cell death and function loss during storage or shipping before the administration to a recipient. In other embodiments, this invention also provides the MSC-containing pharmaceutical product comprising said carrier solution and MSC for use in the treatment of medical conditions in animals and human.

The phrase “Carrier solution” as used herein means a pharmaceutically-acceptable liquid comprising a solvent with or without supplementing additional excipient or material in carrying or transporting the active components (in this invention MSC), in the finished pharmaceutical products from one organ, or portion of the body, to another organ, or portion of the body. Each carrier must be “acceptable” in the sense of being compatible with the other components or ingredients of the formulation of the finished pharmaceutical products and not injurious to the patient.

The carrier solution of this invention is used to resuspend the MSC and maintain the single cell suspension status, viability and biological functions during storage or shipping before administration to recipient(s). MSC are able to secret many adhesive molecules known as extracellular matrix and tend to stick together and form clumps if placed in a steady condition for a prolonged period. Therefore, the carrier solution in our invention comprise at least three components including a balanced salt solution, a low molecular weight heparin and human serum albumin to mitigate or prevent the aggregation of MSC and loss of viability and biological functions in an appropriate temperature.

A balanced salt solution is a solution made to a physiologi-cal pH and isotonic salt concentration. In several embodiments, the balanced salt solution is an isotonic solution which maintains pH and osmotic balance as well as provides cells with water and essential electrolytes. The balanced salt solution closely mimics human plasma in its content of electrolytes, osmolality, and pH and is wildly used to maintain cells for the short-term in a viable condition while the cells are manipulated outside of their regular growth environment or physical conditions. The balanced salt solution in this invention preferably is an injectable grade solution. Examples include: 1) PLASMA-LYTE A Injection pH 7.4 (Multiple Electrolytes Injection, Type 1, USP); 2) 5% Dextrose and Electrolyte No. 48 Injection (Multiple Electrolytes and Dextrose Injection, Type 1, USP); 3) 0.9% Sodium Chloride Injection, USP; 4) Lactated Ringer's Injection, USP.

In the past it has been assumed that the cell death or apoptosis occurs in vitro is mainly related with the nutrient and other supplements in the solution under an appropriate temperature and what has not been realized is that the extent of the formation of cell aggregate may also significantly comprise the cell viability during storage, as the cells in the center or inner space of an aggregate may not get adequate access to the nutrients or oxygen. Applicants have recognized this problem, especially in the case of MSC, a type of cells with high tendency to form aggregation while placed in storage for a prolong period. Applicants have realized that the maintained single cell status is important to mitigate the loss of cell viability for MSC product.

It is also recognized that the single cell status of MSC product is also preferred or required in certain circumstances for clinical use. For example, when MSC product is used systemically via intravenous infusion, the MSC solution needs to be administrated via a blood transfusion kit with filters to eliminate cell aggregates before infused to the blood system of a recipient. A single cell suspension of MSC product can prevent the cell loss during the transfusion.

Low molecular weight heparin is a class of an anticoagulant which consists of only short chains of natural heparin and has an average molecular weight of less than 8000 Da. At least 60% of all chains have a molecular weight less than 8000 Da. Low molecular weight heparin, due to its more predictable anticoagulant effects that natural heparin, are commonly used to prevent blood clots and treatment of venous thromboembolism. The mechanism of action of low molecular weight heparin to inhibit coagulation process is through binding to antithrombin and accelerate its inhibition of activated factor X, a critical protein in the blood coagulation cascade. The anticoagulant effect of low molecular weight heparin occurs both in vivo and in vitro, thus it is also used to prevent blood clotting in the preparation of blood samples. MSC are able to secret many adhesive molecules known as extracellular matrix and tend to stick together and form clumps if placed in a steady condition for a prolonged period. While in many circumstances, MSC pharmaceutical products require the cells in a single cell status (i.e., not in aggregation or in the form of cell clumps) for optimal therapeutic effects. Applicants have found heparin are also able to inhibit the aggregation of MSC in vitro, although the mechanism of action for heparin's inhibition effect on MSC is not clear. The mechanism of action for heparin's inhibition effect on MSC aggregation is presumably different to that on blood coagulation due to the absence of blood coagulation cascade components in MSC suspension in vitro. There are several low molecular weight heparin types due to the different salts, several examples include: Dalteparin sodium, Enoxaparin sodium, and Nadroparin calcium.

Heparin activity (concentration) is measured in either International Units (IU) defined by the World Health Organization (WHO) International Standard, or United States Pharmacopeia (USP) units. There is a small (7-10%) difference between the IU and USP unit. In several embodiments, the carrier solution for MSC product comprises the low molecular weight heparin at the concentration of 5 IU/mL to 100 IU/mL. Thus, according to several embodiments, the final concentration of the low molecular weight heparin in the MSC pharmaceutical product is set at 5 IU/mL, 10 IU/mL, 15 IU/mL, 20 IU/mL, 30 IU/mL, 35 IU/mL, 40 IU/mL, 45 IU/mL, 50 IU/mL, 55 IU/mL, 60 IU/mL, 65 IU/mL, 68 IU/mL, 70 IU/mL, 75 IU/mL, 80 IU/mL, 85 IU/mL, 90 IU/mL, 95 IU/mL, 100 IU/mL or any other concentration between any of these figures.

The carrier solution for MSC product comprises the human serum albumin at the concentration of 0.5% to 5% (v/v.). The final concentration of human serum albumin in the MSC pharmaceutical product is set at 0.5%, 1%, 1.5%, 2.0%, 2.5%, 3.5%, 4.0%, 4.5%, 5%, or any other concentration between any of these figures.

In several embodiments, provided compositions for the carrier solution is to maintain the viability and biological functions of MSC during the preparation, storage and/or shipping of a finished MSC pharmaceutical product which comprises the carrier solution and MSC. “Maintain” when used to refer to a stem cell's viability, means minimal loss of the number of living cells and minimal increase of apoptotic or necrotic cells after a certain period of time compared to the zero time point data. When used to refer to a stem cell's biological function, “Maintain” means retaining, or inhibiting the loss of, a stem cell's anti-inflammation, immunomodulation, secretion of protein, migration, homing, engraftment or differentiation characteristics.

Provided herein are also methods for preparing a finished MSC pharmaceutical product by resuspending the MSC in the carrier solution with said compositions and filling the resuspended MSC-carrier solution mixture into a sterile infusion bag. Several examples of the sterile infusion bag for a finished MSC pharmaceutical product include: CS250, CS500 or CS750 from OriGen Biomedical Inc.

The MSC tissue sources comprise preferably human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

In several embodiments, the MSC containing tissues may be taken from human bone marrow, adipose tissues, blood, amniotic fluid, dental pulp and placenta or other tissues that MSC can be isolated.

In several embodiments, the concentration of MSC in the finished MSC pharmaceutical products is between 1×10⁵ to 1×10⁷ cells per milliliter. The final concentration of MSC in the pharmaceutical product is set at 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷ cells per milliliter or any other concentration between any of these figures.

In several embodiments, the finished MSC pharmaceutical products are stored at 2-8° C. and may be provided as therapies to a patient suffering from a medical disease.

In several embodiments, the finished MSC pharmaceutical products may be used to treat autoimmune diseases or liver diseases, or cancer.

In one embodiment, the finished MSC pharmaceutical products are provided for use in a variety of autoimmune diseases and disorders. As used herein, the term “autoimmune diseases and disorders,” refers to a condition that body's immune system attacks healthy cells and impairs the normal functions of the attacked tissues or organs, such as, for example, rheumatoid arthritis where the body's immune system attacks the joints and causes tender, swelling and function impairment of the joints; Crohn's disease where the body's immune system attacks the digestive tract and causes abdominal pain, diarrhea, fever, and weight loss; multiple sclerosis where the body's immune system attacks the oligodendrocytes of the myelin that covers nerve fibers, thus causing neurologic dysfunctions. Other autoimmune diseases and disorders include, but are not limited to, type 1 diabetes, psoriasis, systemic lupus erythematosus (lupus), Addison's disease, graves' disease, Sjögren's syndrome, Hashimoto's thyroiditis, myasthenia gravis, vasculitis, pernicious anemia, celiac disease.

In another embodiment, the finished MSC pharmaceutical products are provided for use in a variety of liver diseases and disorders. As used herein, the term “liver diseases and disorders,” refers to any conditions that damage the liver and/or prevent it from normal functioning, including but not limited to, cirrhosis where chronic liver damage from a variety of causes leading to scarring and liver failure; alcoholic hepatitis where liver inflammation caused by drinking too much alcohol, for example. Other liver diseases and disorders include, but are not limited to, non-alcoholic fatty liver disease and infectious hepatitis (e.g., hepatitis B).

In another embodiment, the finished MSC pharmaceutical products are provided to treat a variety of malignant diseases. As used herein, the terms “malignant diseases”, “cancer” or “tumor” refer to any diseases in which abnormal cells divide uncontrollably and destroy body tissues. The malignant diseases include, but are not limited to, breast cancer, colon cancer, lung cancer, liver cancer, brain cancer and gastric cancer.

In some embodiments, the therapeutics of the stem cell in this invention is administered to the subject systemically or locally. As used herein, the term “systemically,” for administration is intended to deliver the stem cell therapeutics into the circulatory system so that the entire body will be affected. Common routes for systemic administration include, but are not limited to, intravenous infusion, intra-arterial infusion, portal vein injection, intraperitoneal injection and intranasal injection. The term “locally,” for administration is intended to deliver the stem cell solution to specific site or sites of the body other than the circulatory system. Common routes for local administration include, but are not limited to, intrathecal injection, intra-articular injection, intra-cerebral injection and any routes of injection directly into a tissue or organ parenchyma.

### EXAMPLES

### Example 1: Study on the Effects of HSA Concentrations in Carrier Solutions on MSC Viability

The hUC-MSC product is formulated as an injectable product. Additionally, the cell viability and single cell suspension are important for the activity and potency of the fresh hUC-MSC cell in the MSC suspension.

Cell viability is the key factor affecting the functional activities of hUC-MSC. After formulation, the MSC suspension will be stored at 2-8° C. before administration to the patient. It has been recognized that the cell viability in the MSC suspension will gradually decrease as the storage time increases. Thus excipient HSA was selected to reduce the decreasing rate of the cell viability.

The carrier solution consists of Plasma-Lyte A, heparin calcium with a concentration at 68 IU/mL, and HSA. The carrier solutions with different HSA concentrations were compared. Briefly, MSC suspensions were formulated in carrier solutions with 1% HSA or 2% HSA. A carrier solution without HSA was used as a control. Cell viability was tested for the cell suspensions stored at 2-8° C. for 24, 48 and 72 hours after formulation.

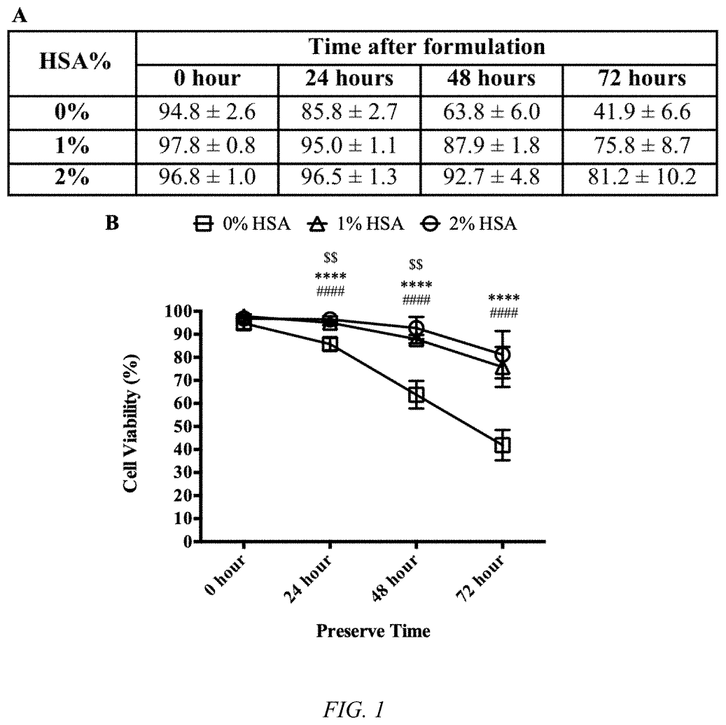

The results showed that the cell viability of cell suspensions with HSA was significantly higher than that without HSA 24, 48 and 72 hours after formulation. Also the cell suspension showed significantly higher cell viability in carrier solution with 2% HSA compared to that in carrier solution with 1% HSA 24 and 48 hours after formulation. (FIGS. 1A and 1B)

### Example 2: Comparison on the Effects on MSC Suspension from the Heparins and Balanced Salt Solutions Used in the Carrier Solutions

In order to minimize the possible side effects of embolism and also maintain optimal MSC population and cell viability, hUC-MSC in the cell suspension are expected to maintain a single cell suspension without obvious cell aggregation. Thus it is critical to select low molecular weight heparins (e.g., heparin sodium and heparin calcium) and proper balanced salt solutions (e.g., Plasma-Lyte A and lactated Ringer's solution) to maintain a MSC population of single cell in a good condition.

Therefore, hUC-MSC suspension (1×10⁶ cells/mL) were formulated in Plasma-Lyte A with heparin calcium or heparin sodium of different concentrations (0, 5, 10, 20, 40 and 68 IU/mL) as well as in lactated Ringer's solution with heparin sodium of different concentrations (0, 5, 10, 20, 40 and 68 IU/mL). All carrier solution contain 2% HSA. All samples were stored at 2-8° C. for 24 hours after formulation. MSC population (%) per flow cytometric event, single cell population (%) and cell viability (%) were measured using a flow cytometry assay.

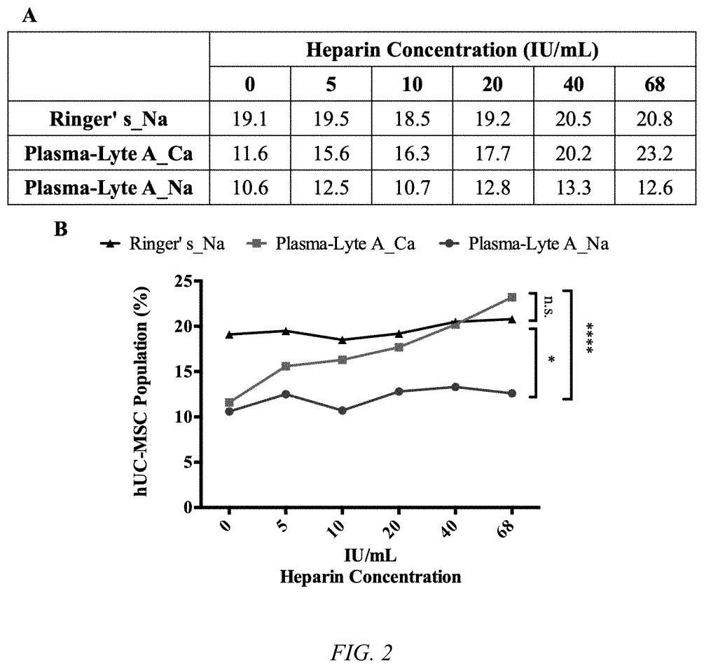

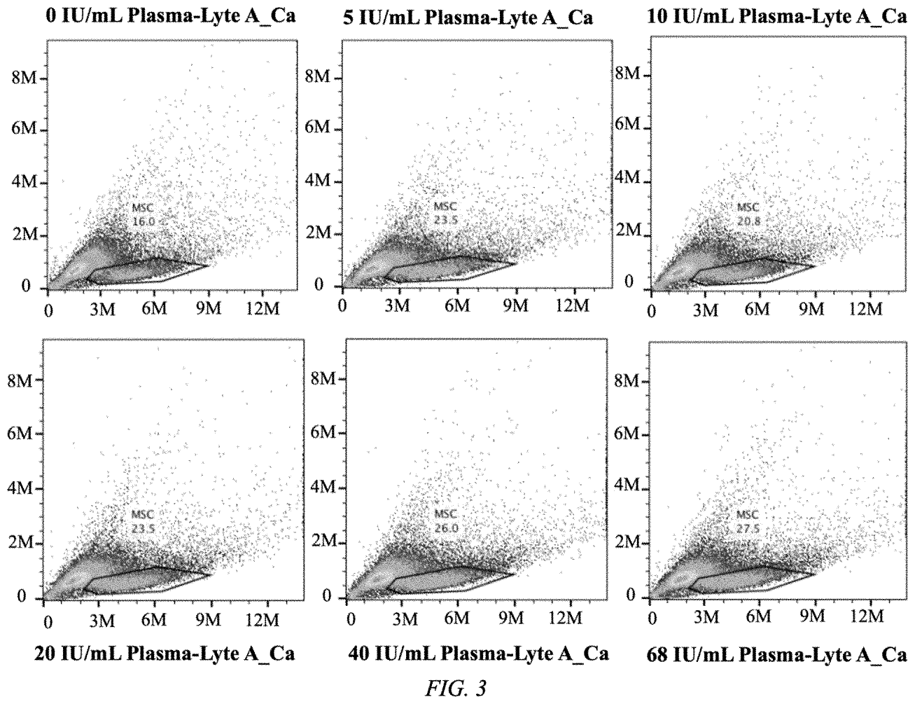

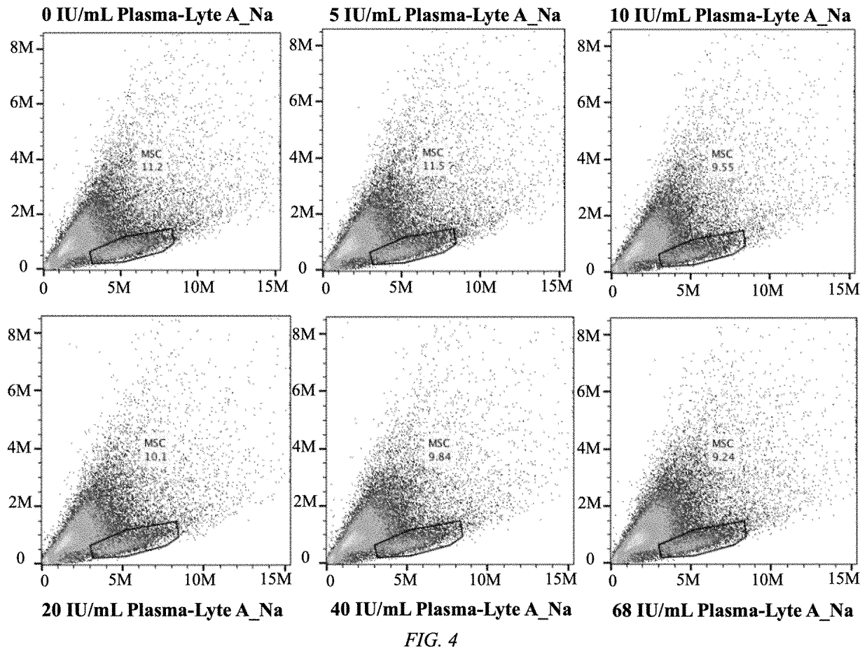

MSC population per event was significantly higher in Plasma-Lyte A with heparin calcium (****p<0.0001) compared to that in Plasma-Lyte A with heparin sodium (FIGS. 2A-2B, FIG. 3 and FIG. 4). Intriguingly, the MSC population (%) tended to increase as the calcium concentration went up (FIG. 3) whereas that remained at a similar level with different concentrations of sodium heparin (FIG. 4). The results indicate that calcium concentration may serve an important role in maintaining MSC population in cell suspension.

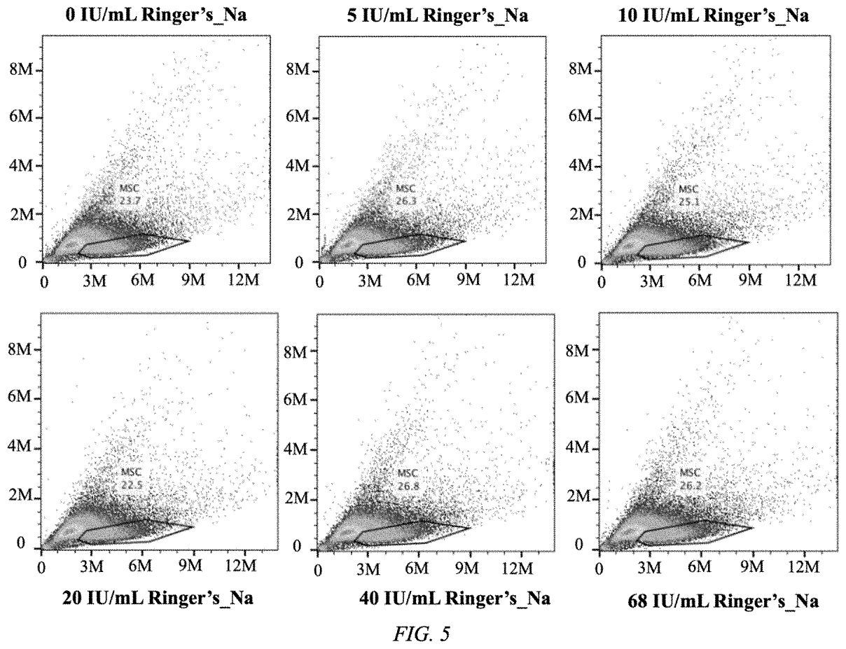

With different concentrations of heparin sodium, MSC population per event was significantly higher in lactated Ringer's solution (*p<0.05) compared to that in Plasma-Lyte A (FIGS. 2A-2B, FIG. 4 and FIG. 5). The MSC population (%) remained at a similar level with different concentrations of sodium heparin (FIG. 2B, FIG. 4 and FIG. 5). The results indicate that lactated Ringer's solution may promote maintaining MSC population in cell suspension.

Importantly, no significant difference of MSC population per event was observed between cell suspension in lactated Ringer's solution with heparin sodium and Plasma-Lyte A with heparin calcium, both of which showed superior effects on maintaining MSC population compared to that in Plasma-Lyte A with heparin sodium (FIGS. 2A-2B, FIG. 3, FIG. 4 and FIG. 5). These results suggested that lactated Ringer's solution with heparin sodium and Plasma-Lyte A with heparin calcium could be equivalent to serve as carrier solutions to formulate MSC suspension.

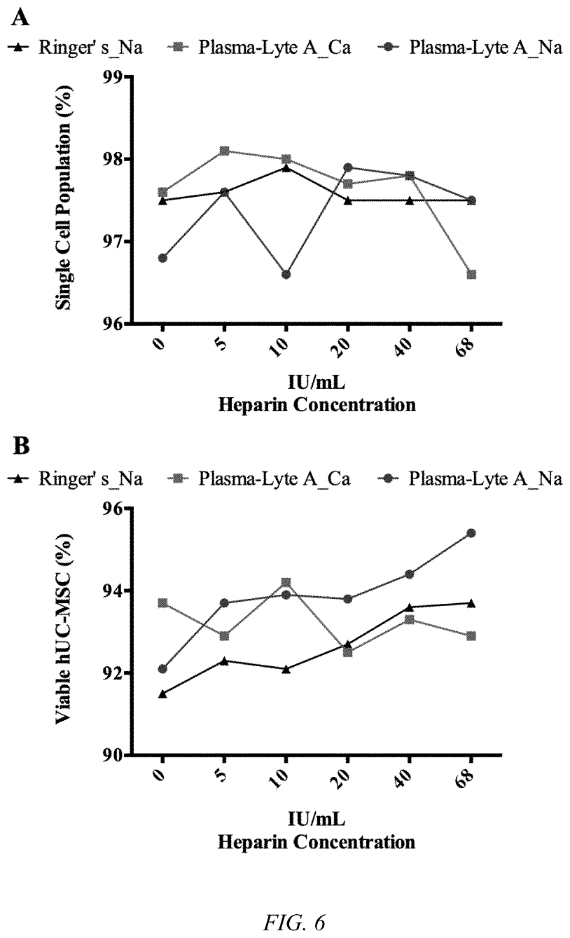

No significant difference on single cell population (%) and cell viability (%) was observed between cell suspensions in lactated Ringer's solution with heparin sodium and Plasma-Lyte A with heparin calcium (FIGS. 6A-6B). This further indicated the equivalence between the two carrier solutions.

### REFERENCES

Chinnadurai, R., I. B. Copland, M. A. Garcia, C. T. Petersen, C. N. Lewis, E. K. Waller, A. D. Kirk, and J. Galipeau. 2016. ‘Cryopreserved Mesenchymal Stromal Cells Are Susceptible to T-Cell Mediated Apoptosis Which Is Partly Rescued by IFNgamma Licensing’, Stem Cells, 34: 2429-42.
Crisan, M., S. Yap, L. Casteilla, C. W. Chen, M. Corselli, T. S. Park, G. Andriolo, B. Sun, B. Zheng, L. Zhang, C. Norotte, P. N. Teng, J. Traas, R. Schugar, B. M. Deasy, S. Badylak, H. J. Buhring, J. P. Giacobino, L. Lazzari, J. Huard, and B. Peault. 2008. “A perivascular origin for mesenchymal stem cells in multiple human organs”, Cell Stem Cell, 3: 301-13.
Francois, M., I. B. Copland, S. Yuan, R. Romieu-Mourez, E. K. Waller, and J. Galipeau. 2012. “Cryopreserved mesenchymal stromal cells display impaired immunosuppressive properties as a result of heat-shock response and impaired interferon-gamma licensing”, Cytotherapy, 14: 147-52.
Moll, G., J. J. Alm, L. C. Davies, L. von Bahr, N. Heldring, L. Stenbeck-Funke, 0. A. Hamad, R. Hinsch, L. Ignatowicz, M. Locke, H. Lonnies, J. D. Lambris, Y. Teramura, K. Nilsson-Ekdahl, B. Nilsson, and K. Le Blanc. 2014. “Do cryopreserved mesenchymal stromal cells display impaired immunomodulatory and therapeutic properties?”, Stem Cells, 32: 2430-42.
Wang, L., L. Wang, X. Cong, G. Liu, J. Zhou, B. Bai, Y. Li, W. Bai, M. Li, H. Ji, D. Zhu, M. Wu, and Y. Liu. 2013. “Human umbilical cord mesenchymal stem cell therapy for patients with active rheumatoid arthritis: safety and efficacy”, Stem Cells Dev, 22: 3192-202.
Wang, L., Y. Zhang, H. Li, J. Hong, X. Chen, M. Li, W. Bai, J. Wang, Y. Liu, and M. Wu. 2016. “Clinical Observation of Employment of Umbilical Cord Derived Mesenchymal Stem Cell for Juvenile Idiopathic Arthritis Therapy”, Stem Cells Int, 2016: 9165267.

What is claimed is:

1. A carrier solution for mesenchymal stem cells (MSC) for pharmaceutical use comprising:
A balanced salt solution,
a low molecular weight heparin, and
a human serum albumin.

2. The carrier solution of claim 1, wherein the balanced salt solution is a lactated Ringer's solution, a 0.9% saline (sodium chloride), or a multiple electrolyte injection.

3. The carrier solution of claim 2, wherein the multiple electrolyte injection is Plasma-Lyte A.

4. The carrier solution of claim 1, wherein the low molecular weight heparin is low molecular weight heparin sodium or low molecular weight heparin calcium.

5. The carrier solution of claim 1, wherein the final concentration of the low molecular weight heparin is between 5 to 100 IU/mL.

6. The carrier solution of claim 1, wherein the final concentration of the human serum albumin is between 0.5% to 5% (V/V).

7. A method for preparing an MSC-containing pharmaceutical product comprising:
providing a tissue source for mesenchymal stem cells (MSC);
isolating the MSC from the issue source;
providing a carrier solution; and
suspending the MSC in the carrier solution,
wherein the carrier solution comprises a balanced salt solution, a low molecular weight heparin, and a human serum albumin.

8. The method of claim 7, wherein the tissue source is an umbilical cord tissue, a bone marrow, an adipose tissue, a blood, an amniotic fluid, a dental pulp, or a placenta.

9. The method of claim 7, wherein the tissue source has a concentration of between 1×10⁵ to 1×10⁷ cells per milliliter of solution.

10. A method for treating a medical disease comprising:
administering to a patient in need thereof an MSC-containing pharmaceutical product,
wherein the MSC-containing pharmaceutical product comprises mesenchymal stem cells (MSC) and a carrier solution; and
wherein the carrier solution comprises a balanced salt solution, a low molecular weight heparin, and a human serum albumin.

11. The method of claim 10, wherein the method is for treating an autoimmune disease, a liver disease, or a cancer.

12. The method of claim 11, wherein the autoimmune disease is Crohns' diseases, ulcerative colitis, multiple sclerosis, rheumatoid arthritis, systemic lupus erythematosus, autoimmune pancreatitis, Type 1 diabetes, or systemic sclerosis.

13. The method of claim 11, wherein the liver disease is liver failure, cirrhosis, hepatic steatosis, or hepatitis.

14. The method of claim 10, wherein the MSC-containing pharmaceutical product is administered intravenously, intraperitoneally, intrathecally, or locally to a specific tissue/site of action.

15. The method of claim 10, wherein the MSC is isolated from a tissue source selected from the group consisting of an umbilical cord tissue, a bone marrow, an adipose tissue, a blood, an amniotic fluid, a dental pulp, and a placenta.
